(19) United States
(12) Patent Application Publication
(10) Pub. No.: US 2008/0160068 A1
(43) Pub. Date: Jul. 3, 2008

(54) Method for a Medicinal Combination Treatment, and Medicament Combinations Suitable Therefor

(75) Inventors: Thomas Hille, Neuwied (DE); Werner Wessling, Rengsdorf (DE)

Correspondence Address: D. PETER HOCHBERG CO. L.P.A., 1940 EAST 6TH STREET, CLEVELAND, OH, 44114, US

(73) Assignee: LTS Lohmann Therapie-Systeme AG, Andernach (DE)

(21) Appl. No.: 11/884,797

(22) PCT Filed: Feb. 14, 2006
(86) PCT No.: PCT/EP2006/001312

§ 371 Date: Aug. 21, 2007

(30) Foreign Application Priority Data
Feb. 21, 2005 (DE) 10 2005 007 859.1

(51) Int. Cl. A61K 9/70

(52) U.S. Cl. 424/449

(57) ABSTRACT

A method for the administration of at least one pharmaceutical agent to a patient who depends on the administration of the agent or agents, comprising the application of at least one transdermal therapeutic system containing a first pharmaceutical agent for the transdermal administration of the agent during a predeterminable period and the application of at least one wafer at the beginning or during the period of the transdermal administration. The wafer contains the same agent or a second or further agents suitable for the same indication as the first agent.

### CROSS-REFERENCE TO RELATED APPLICATIONS

This application is a National Stage application of International Application No. PCT/EP2006/001312, filed on Feb. 14, 2006, which claims priority of German application number 10 2005 007 859.1, filed on Feb. 21, 2005.

### BACKGROUND OF THE INVENTION

1. Field of the Invention

This invention relates to methods for administration of pharmaceutically active substances to patients who depend on the administration of such pharmaceutically active substances, for the purpose of a medicinal treatment, particularly for carrying out a long-term therapy. The invention furthermore relates to combinations of medicaments which are suitable for such methods, and to the use of pharmaceutically active substances in such therapeutic methods.

The invention particularly relates to a combination treatment by transdermal therapeutic systems and simultaneous administration of one or more wafers for the transmucosal release of active substance(s) in the oral cavity.

2. Description of the Prior Art

The combination therapy according to the invention can be advantageously used with in the therapeutic treatment of patients in need of a long-term therapy or basic medication with a pharmaceutically active substance which continues for a prolonged period, and where in addition thereto there is, at the beginning or during the long-term therapy, a necessity of bringing about a rapid or accelerated onset of medicament action and/or of treating a temporary, increased active substance requirement which occurs unexpectedly during the long-term therapy.

This problem is significant, in particular, in the treatment of severe or chronic pain (e.g., in the pain therapy of tumour patients), where to achieve a lasting relief from pain, a long-term or basic therapy with analgesics (e.g. opioids) is carried out wherein the administered dose is adjusted to the intensity of pain perceived by the patient. The aim of such a therapy is to sufficiently reduce the pain while avoiding overdosage.

However, during such a long-term or basic therapy with an individually adjusted active substance dose, so-called breakthrough pain may occur. The term “breakthrough pain” refers to pain that occurs temporarily during or despite continual intake of analgesics according to the time schedule (long-term medication) and which is more intense than the pain treated by the long-term therapy. Frequently, breakthrough pain is triggered by a factor that can be identified and thus avoided, for example voluntary movements, certain body postures, contact, or, in the case of long-term pain in the gastro-intestinal region, certain foods. To treat these breakthrough pains, the patient has to have a further analgesic prescribed. In addition to the analgesic administered within the framework of the basic therapy, the latter generally being a controlled-release preparation.

The transdermal application of medicinal substances (pharmaceutical active substance), particularly by transdermal therapeutic systems (TTSs), has a number of advantages which are generally known, for example a controlled and long-lasting delivery of active substance and the avoidance of the “first pass effect”. However, vis-à-vis these advantages there frequently is the disadvantage that the uptake of medicinal substances via the skin is limited both with regard to quality and quantity and that the absorption of active substance through the skin following application of a TTS on the skin starts only after a long delay in time.

It is known to those skilled in the art that the skin is not an absorptive organ, but rather has the function of preventing the intrusion of foreign bodies and thereby also of medicinal substances. Thus, it is this property of the skin which is responsible for the above mentioned delay of the onset of action. For this phenomenon, the term “lag time” has been coined. This term is understood to mean the time between the first application of a transdermally applicable medicament, for example a TTS, and the first occurrence of a measurable plasma concentration or the first occurrence of the expected physiological effect of the pharmaceutic.

This “lag time” is particularly critical in cases where a medicinal substance is to be applied not only for the purpose of a basic or long-term therapy, that is, for a prolonged period, but where, in addition, there is a demand that the action of that medicinal substance occur as immediately after the first application of the medicament as possible, for example when applying centrally active analgesics. It is true that when a TTS is applied for the first time or when breakthrough pain occurs it is possible to avoid or shorten the disadvantageous “lag time” by additionally administering a medicament which exhibits a rapid delivery of active substance, for example an intravenous injection. However, such a combined application is not without problems since an intravenous injection must always be given by a physician. Administration of tablets with simultaneous application of TTSs is not helpful either since the gastro-intestinal absorption of opiates also occurs only with some delay.

In addition, the administration of tablets results in the active substance, after its gastrointestinal passage, passing through the liver, where it is metabolised, that is, rendered inactive. To those skilled in the art this phenomenon is known as the so-called “first pass effect”. Particularly with opiates containing a free hydroxyl group on an aromatic ring of the morphinan skeletal structure (e.g. morphine and hydromorphone), the chemical Phase II conversion, i.e. glucuronidation (conjugation with glucuronic acid), starts early.

Because of the above described disadvantageous (delayed onset of action) transdermal administration is not suited for the treatment of pain which occurs suddenly, for example breakthrough pain. When the development of a therapy by dermal or transdermal application began, attempts were made at the same time to find methods by which the “lag time” could be shortened and the onset of action accelerated.

One possibility of accelerating the transdermal active substance absorption is to treat the TTS, which has been applied to the skin, with ultrasound or by the development of heat. The drawback of these methods is that their practical implementation is difficult. They have therefore not prevailed in practice.

Other methods for increasing the absorption rate of medicinal substances in transdermal administration are based on removing or partially damaging the stratum corneum of the skin by laser treatment or by repeatedly sticking on and tearing off an adhesive strip (so-called “stripping”). Although these two treatment methods likewise shorten the “lag time”, these methods are disadvantageous in that they do not only facilitate the desirable penetration of the medicinal substance, but also facilitate the undesirable intrusion of other components of the medicament and of microorganisms, such as bacteria or fungal spores, into the human body. The method furthermore has the disadvantage that in order to “strip” the skin, the TTS must be removed. However, as is known to medical experts, peeling away a TTS leads to loss of adhesion since the uppermost skin layer, which is in contact with the adhesive, is removed along with the TTS.

Another way of improving the dermal absorption rate is to use electric current. As is known to medical experts, this method, known under the term “iontophoresis”, cannot be applied without causing pain. The same is true of the so-called spiked patch. This form of dermal medicament is fixed to the body by cannulae which penetrate the skin. Active substance delivery takes place via the cannulae, which at the same time serve as fixation aids. It is obvious that this can no longer be called a dermal or transdermal application in the classical sense of the word, but is in fact a subcutaneous injection of a medicinal substance, with all its known disadvantages (i.e., necessity of sterile cannulae, no protracted release, etc.).

### SUMMARY OF THE PRESENT INVENTION

It was therefore the object of the present invention to provide a therapeutic method which enables the administration of a medicinal substance to a patient for carrying out a basic or long-term therapy, i.e. over a prolonged period of time, and which reduces or avoids the aforementioned disadvantages (particularly the “lag time” and the “first pass” effect). More particularly, the object was to provide a method of medicinal treatment which enables the initiation or maintenance of a basic or long-term therapy, and where the therapeutic action is to commence as immediately after the first administration as possible. In other words, the “lag time” is to be minimised.

Another object of the invention was to indicate methods of treatment by which it is made possible to administer at least one additional dose of medicinal substance with a “lag time” that is as short as possible, in periods which occur during long-term therapy and which are characterized by an increased medicinal substance requirement of the respective patient.

Furthermore, it was an object of the invention to provide means that are suitable for carrying out the above-mentioned methods.

These objects are achieved according to the present invention by administrating at least one pharmaceutically active substance to a patient who depends on the administration of the at least one pharmaceutically active substance for carrying out a long-term therapy, as well as by the products and therapeutic uses according to the present invention.

### BRIEF DESCRIPTION OF THE FIGURE

FIG. 1 shows a graph providing medium plasma levels on the y-axis and time on the x-axis.

### DETAILED DESCRIPTION OF THE PRESENT INVENTION

Thus, the present invention relates to a method of administering at least one pharmaceutically active substance to a patient who depends on the administration of the active substance or active substances. More particularly, this method is a method for carrying out a long-term therapy.

The method of treatment according to the invention comprises (a) the application of at least one transdermal therapeutic system (TTS), containing a first pharmaceutically active substance, for the transdermal administration of the active substance during a predeterminable period of time, and (b) the application of at least one wafer at the beginning of or during the period of time of the transdermal administration, wherein the wafer contains the same active substance or a second or further active substances suitable for the same indication as the first active substance.

By applying a TTS, the active substance dose required for initiating or/and maintaining a long-term therapy is provided and is delivered, with a delayed, controlled release, to the skin of the patient and made systemically available. The period of time of the transdermal administration depends essentially on the total amount of active substance contained in the respective TTS, on the type of active substance contained, on the delivery surface area of the TTS, and on the release rate. Generally, the release period of a TTS applied to a patient's skin is in the range from approximately 6 to 72 hours, particularly 12 to 24 or 48 hours. After this predeterminable time, the spent TTS is removed and, if necessary, replaced by a new TTS. The overall duration of a long-term therapy may be one or several days, or may extend over an indefinite period of time, as long as the indication persists.

The wafer mentioned in (b) is a wafer-shaped, thin and pliable administration form which is preferably applied orally and which releases the active substance(s) contained therein in the oral cavity, with the active substance absorption taking place mainly via the oral mucosa (i.e., transmucosally). Because of the small thickness of these wafers (preferably 0.05 to 1 mm, especially 0.1 to 0.5 mm), and the short diffusion paths, the release of active substance starts immediately after the wafer has been introduced in the oral cavity. Due to the transmucosal absorption, a therapeutically effective plasma level is achieved within a few minutes (approximately 5 to 15 minutes) following the oral administration of a wafer. This enables a rapid onset of action. Preferably, wafers are used that are mucoadhesive or/and are disintegratable in aqueous media (i.e., body fluids, especially saliva).

The wafer is applied according to (b) at the beginning or during the period of time of the transdermal administration. This means that the wafer is applied at the beginning of transdermal administration (i.e. at the time of applying a TTS to the skin), or that the wafer is administered to the patient at a later time, when the TTS is already on the patient's skin and the transdermal delivery of active substance has already begun.

In the simplest case, a wafer which is applied at the beginning of or during the period of time of the transdermal administration contains the same active substance or the same combination of active substances as the TTS by which the transdermal administration is achieved. As an alternative, or in addition thereto, such a wafer may contain a second or further active substances which is/are not identical with the (first) active substance contained in the TTS, but which is/are suitable for the same indication as the first active substance. This active substance may be a medicinal substance having the same pharmacological activity. In the case of analgesics this may be another opioid, for example. If the TTS contains a combination of two or more active substances, the wafer which according to (b) is administered in addition to the TTS, may optionally contain only one of the active substances of that active substance combination.

By combining, in accordance with the invention, a transdermal administration with the administration of one or several wafer(s), it is now made possible to carry out a long-term therapy which is characterized by a rapid onset of action and which enables rapid dose adjustment when, during the long-term therapy, phases of illness occur in which the active substance requirement is temporarily increased, particularly for treating breakthrough pain in long-term pain therapy. Thus, the methods according to the present invention are preferably suitable for those patients where a rapid or accelerated onset of therapeutic action is required, either at the beginning of a long-term therapy or during the period of a long-term therapy, or for the treatment of patients where during the long-term therapy or the basic medication there occurs a temporarily increased active substance requirement.

Therefore, according to a preferred embodiment of the invention, in a first step of the treatment method, a TTS which contains a first active substance is applied and, in addition thereto, a wafer is applied which contains the same active substance or a second or further active substances suitable for the same indication. Preferably, the TTS and the wafer are applied almost simultaneously, that is, within a period of less than 15 minutes, preferably less than 5 minutes. The TTS applied to the skin remains on the skin for the predetermined period (e.g. 12 to 72 hours) in order to provide the basic therapy.

The additional administration of a wafer, as described above, may preferably be performed once, at the start of a basic therapy. If necessary, one or more further wafers may be applied during the further course of the long-term therapy.

To maintain the basic therapy according to requirements for a prolonged period of time, further transdermal therapeutic systems containing the active substance may be administered to the patient at regular intervals (e.g., after 6, 12, 24, 48 or 72 hours), each time removing the respective previously applied, spent, TTS from the skin. In this way it is possible to continue the long-term therapy or basic therapy for a prolonged period of time, preferably for at least 24 hours. The long-term therapy may be continued for a period of several days, weeks, months or years, if required by the circumstances of the disease.

According to a further, preferred embodiment of the invention, the method of treatment comprises at least one step wherein a transdermal therapeutic system is administered jointly with a wafer, as described above. This joint application may preferably take place at the beginning of the treatment (especially at the beginning of a long-term or basic therapy). Alternatively, it is possible to apply a wafer simultaneously with each successive application of a further TTS.

Another, particularly preferred embodiment of the invention, provides that during the above-mentioned period of time of the transdermal administration or during the long-term therapy there is at least once an additional administration of the active substance or of another active substance which is suitable for the same indication, in the form of a wafer. The active substance dose administered by the wafer enables the treatment of an increased active substance requirement of the patient which occurs temporarily during the period of time. In particular, it is thereby made possible to treat breakthrough pain or peaks of pain occurring during a long-term pain therapy. The rapid systemic availability of the active substance administered transmucosally by the wafer results in a quick alleviation of the pain. Application of the wafers used in accordance with the invention may be performed in a simple manner by the patient himself. When required—for example when particularly intense breakthrough pain occurs—two or more wafers may be administered simultaneously or at short time intervals.

The amount of active substance (“acute dose” or “bolus dose”) contained in a wafer according to the present invention, which is administered, for example, upon initiation of a long-term therapy or for treating breakthrough pain, preferably corresponds to 0.1 to 0.7 times, especially preferably 0.2 to 0.5 times, the transdermally administered daily dose.

Preferably, the methods of the invention are applied for treating patients who suffer from one or more of the following diseases, conditions or symptoms: chronic pain, asthma, diabetes, risk of cardiac infarction, nicotine withdrawal and Parkinson's disease. In a particularly preferred embodiment of the method, the method is used for the treatment of pain. This pain may be chronic and/or acute conditions of pain, as occurring, for example, in tumour patients.

Medicinal substances which are suitable for treating the aforementioned diseases, conditions or symptoms are known to those skilled in the art. Active substances selected from the group which comprises analgesics, broncholytics, antidiabetics, vasodilators, withdrawal agents and anti-Parkinson agents are particularly suitable for this purpose.

Generally, all pharmaceutically active substances may be used for the purposes of the present invention which can be applied transdermally since in these cases it is also to be assumed that these active substances are also quickly absorbed via the mucosa of the mouth. If an active substance selected for the transdermal administration has only an insufficient transmucosal absorption rate, this active substance may, as mentioned above, be replaced by another active substance which is suitable for the same indication as the transdermally administered active substance, but exhibits better transmucosal absorption.

Preferably, for transdermal administration, those pharmaceutically active substances are selected which exhibit a low skin penetration rate, so that the intended delayed and long-lasting action is achieved. Alternatively, the rate of active substance release from the transdermal therapeutic system may be controlled in a manner known to those skilled in the art and—if necessary—reduced, for example by auxiliary substances suitable for that purpose, or by control membranes retarding the release of active substance.

Suitable for the purposes of the present invention are, above all, such active substances as are highly efficacious, that is, those active substances the daily dose of which is in the milligram range (e.g., 1 to 500 mg) and the pharmacologically acceptable salts of which are readily soluble in water (preferably exceeding 10%, relative to the mass). This is true, in particular, of opioids and their salts, the use of which is therefore particularly preferred.

In the case of pain therapy, analgesics, preferably those from the group of the opioids, are particularly suitable in connection with the method according to the invention.

“Analgesics” is, for the purposes of this invention, understood to mean medicinal substances which, in therapeutic doses, are suitable for reducing or suppressing the sensation of pain. This includes, in particular, centrally active, highly efficacious analgesics, the so-called opioids. This group of pharmaceutically active substances includes, inter alia, morphine, heroin and other derivatives of morphine; dihydromorphine derivatives such as hydromorphone (dihydrocodeine), oxycodone; morphinan derivatives such as levorphanol, buprenorphine; analgesics of the pethidine group, such as pethidine, ketobemidone, loperamide, diphenoxylate; methadone and derivatives such as levomethadone, dextromoramide, dextropropoxyphene; fentanyl and its derivatives (e.g. alfentanil, sufentanil, remifentanil), benzomorphane derivatives such as pentazocine and phenylaminocyclohexinyl derivatives such as tilidine; tramadol. For the treatment of breakthrough pain, opioids having a rapid and short action, such as morphine, tramadol, tilidine, oxycodone, hydromorphone, buprenorphine, fentanyl and levomethadone, are particularly preferred.

For the purpose of transdermal administration, preference is given to analgesics exhibiting a low skin penetration rate. An example of this is buprenorphine.

Furthermore, the following examples from the group of the analgesics are also suitable: metamizole, phenazone, propyphenazone, flupirtine, nefopam; anti-epileptics such as carbamazepine, gabapentin, clonazepam; anti-depressants such as amitryptiline.

The invention also encompasses the use of active substance combinations consisting of two or more medicinal substances, particularly combinations of the aforementioned analgesics.

It is obvious that the practical application of the present invention is of particular importance for the administration of analgesics since in a state of acute pain it is unacceptable for the patient to wait until the end of the “lag time” for the action of the medicament to commence. In such a case, an acceptable “lag time” would be a period of up to a few minutes (i.e., 5 to 10 minutes). This condition is fulfilled by the use according to the invention of orally applicable wafers for transmucosal active substance administration.

The present invention furthermore encompasses a combination of medicaments which comprises at least one transdermal therapeutic system (TTS) and at least one active substance-containing wafer, wherein the TTS(s) contain(s) a first pharmaceutically active substance, and wherein the wafer(s) contain(s) the same first active substance or a second or further active substances which is/are suitable for the same indication as the first active substance.

The term “medicament” generally refers to substances or substance mixtures for human or veterinary medicine which contain the pharmaceutically active substance(s) as well as further usual components (inactive auxiliary substances) that render the active substance pharmaceutically usable. The inventive combination of medicaments comprises medicaments which are present as different administration forms, namely, on the one hand, in the form of a TTS and, on the other hand, in the form of a wafer.

In the medicament combination according to the invention, a certain number of transdermal therapeutic systems (TTSs) containing the same pharmaceutically active substance and preferably also in other respects being of identical composition, is allocated to a certain number of wafers. These wafers preferably contain the same active substance as the TTS(s), or a different active substance which is suitable for the same indication as the active substance contained in the TTS(s). In the case of the wafers, too, it is preferred that all wafers belonging to a combination are of an essentially identical composition.

The aforementioned allocation of a certain number of TTSs to a certain number of wafers may preferably be realised such that these TTSs and wafers are packed in a joint package and are present as a “set” or “kit”.

A medicament combination according to the invention contains at least one TTS and at least one wafer. The number of TTSs and the number of wafers in a combination may optionally be the same or different. Particularly for use in pain therapy, it is preferred that the wafers be contained in the combination in a number that is larger than that of the transdermal therapeutic systems. The wafers contained in a certain medicament combination usually have the same content of active substance. Besides, it can be advantageous if such a combination contains two or more groups of wafers which differ from each other in terms of their active substance dose and which are correspondingly marked.

The active substances contained in the transdermal therapeutic systems and wafers of the medicament combination are preferably selected from the above-mentioned active substances and active substance groups. It is furthermore preferred that the transdermal therapeutic systems contained in the medicament combination enable a systemic, transdermal administration of the active substances contained therein over a period of at least 24 hours, preferably at least 48 hours. The wafers contained in the medicament combination are preferably wafers for oral administration, wherein the therapeutic action begins at the latest 15 minutes, preferably at least 5 minutes, following oral administration. It is furthermore preferred that the wafers be mucoadhesive or/and disintegratable in aqueous media.

The invention furthermore comprises the use of pharmaceutically active substances, especially of active substances which are selected from the above-mentioned active substances and active substance groups, for the manufacture of the above-described medicament combination according to the invention for treating patients who depend on the administration of such an active substance in order to achieve a long-term therapy or basic therapy. These medicament combinations are preferably used in the above-described therapeutic treatment methods and for the above-mentioned therapeutic purposes.

The TTSs and wafers according to this invention may be manufactured using known pharmaceutical methods. The compositions of these formulations and the auxiliary substances used therein are likewise known to those skilled in the art.

TTSs which may be used for the purposes of the present invention are described, for example, in DE 39 39 376 C1, DE 199 23 551 A1 and DE 198 34 005 A1.

The TTSs used in accordance with the invention preferably have a layered structure, that is, they are two-, three- or multilayered. More particularly, the layered structure of the TTSs may comprise one or more layers selected from pressure-sensitive adhesive layers, porous layers and, hydrogel layers.

At least one of the layers of the TTS contains an active substance or an active substance combination, as described above. Preferably, the TTSs according to this invention are provided with a pressure-sensitive adhesive layer which serves to attach the TTS on the patient's skin and which preferably contains active substance.

The active substance-containing layer (or matrix) of the TTS preferably consists of a pressure-sensitive adhesive, water-insoluble polymer, e.g. partially esterified polyacrylates, polyisobutylene or silicones, or of mixtures of such polymers. In addition, known auxiliary substances can be admixed (e.g. solubilisers, emulsifiers, permeation enhancers, preservatives).

The wafer used in accordance with the invention may, without limiting the invention, be a sheet-like object for oral administration of active substances. In this case, the active substance is present dissolved in a polymer or polymer mixture or dispersed in a polymer matrix. The polymers used for manufacturing the wafer should preferably be water-soluble so that the wafer, as intended, can dissolve quickly, ideally within seconds (e.g. maximally 5 to 30 seconds) in the saliva of the oral cavity.

Suitable polymers for the manufacture of the wafers are, in particular, those polymers from the group of the polyethylene glycols, starch and starch derivatives, polyvinyl alcohols and polyacrylic acid (e.g. CARBOPOL®), or polyvinyl pyrrolidone (polyvidone, e.g., KOLLIDON®). Furthermore, the wafers may contain one or more auxiliary substances such as softeners, emulsifiers, surfactants, solubilisers, fillers, disintegrants, colourants, flavouring substances and sweeteners, preservatives; such auxiliary substances are known to those skilled in the art. Wafers which may be used for the purposes of the present invention and suitable methods for the manufacture thereof are described in DE 102 07 304 A1 and U.S. Pat. No. 6,709,671 B2, for example.

### EXAMPLE

The invention will now be explained in greater detail by the below example.

This example relates to the therapeutic treatment of a pain patient. For long-term treatment or for a basic therapy, a TTS (or several TTSs) containing buprenorphine is/are manufactured, as described in DE 39 39 376 C1 (see the following table). This TTS is applied to the skin of a pain patient and remains there for the intended period of application (e.g. 24, 48 or 27 hours).

The TTS used contains medicinal substances and auxiliary substances according to the following table:

| Medicinal substance or | |
|---|---|
| auxiliary substance | Amount per TTS [mg] |
| Buprenorphine base | 20 |
| Oleyl oleate | 30 |
| Levulinic acid | 20 |
| Polyacrylate adhesive, crosslinked with | 680 |
| aluminium | |
| Polyethylene terephthalate fabric as backing layer | 518 |
| Polyethylene terephthalate film | 80 |
| in 23 μm thickness | |
| Siliconized polyethylene terephthalate film as | 919 |
| protective layer | |

The release rate of such a TTS is 35 μg/h (relating to the release of buprenorphine from a respective single TTS).

Simultaneously, a wafer is administered orally to that patient. This wafer contains 10 percent by weight of buprenorphine hydrochloride in a polymer mixture of 65 percent by weight of CARBOPOL® and 25 percent by weight of starch. A single wafer essentially consists of 1 mg of buprenorphine hydrochloride, 6.5 mg CARBOPOL® and 2.5 mg starch.

The high concentration of the medicament in the wafer allows for the patient to experience an alleviation of pain immediately at the start of the long-term therapy since the wafer disintegrates in the mouth within about 5 to 10 seconds and the buprenorphine which is released (as a water-soluble salt) is absorbed directly via the oral mucosa, so that a therapeutically effective plasma level is achieved within a few minutes.

If during the period of transdermal active substance administration breakthrough pain occurs, one or more of the buprenorphine-HCL-containing wafers are applied in the oral cavity of the patient as early as possible (i.e., as soon as the first signs of an increase in the intensity of the pain is perceived). Due to the rapid absorption of the water-soluble salt via the oral mucosa, the patient can thereby experience immediate relief in an acute condition of pain.

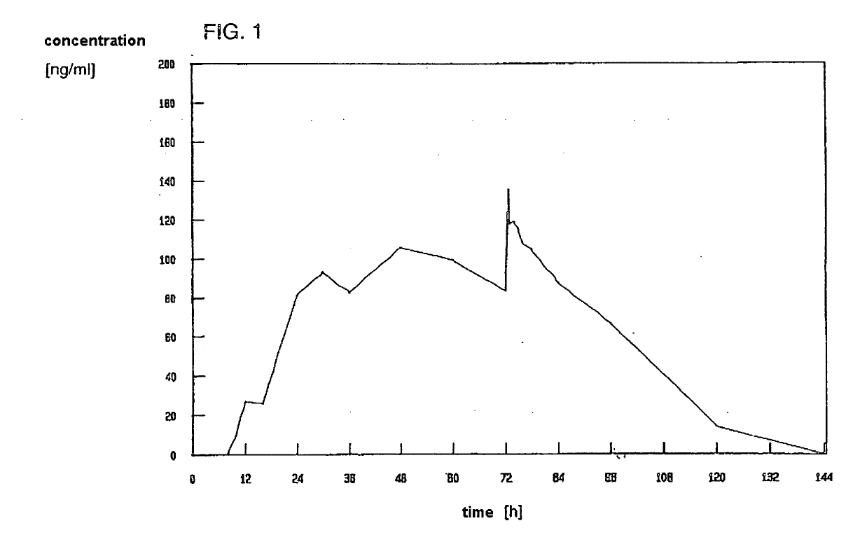

FIG. 1 shows medium plasma levels that were determined by applying the buprenorphine-containing TTS to n=5 healthy volunteers. As can be clearly seen, in the first 12 hours pain patients were not sufficiently treated; the plasma concentration achieved is below 30 ng/ml (“lag time”). It is only after 24 hours that a basic therapy is securely achieved by the plasma plateau (buprenorphine concentration approximately 80-100 ng/ml). This plateau concentration is maintained for a period of up to about 96 hours following application of the TTS.

When breakthrough pain occurs, as well as in the first 12 hours after application of the TTS, through the additional administration, provided for according to the invention, of a wafer for oral application, it is achieved that the action of the buprenorphine commences early and that the “first pass effect” is avoided, so that a rapid and efficient alleviation of the breakthrough pain is effected.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A method for the administration of at least one pharmaceutically active substance to a patient who depends on the administration of said at least one pharmaceutically active substance for carrying out a long-term therapy, said method comprising the steps of:
a) applying at least one transdermal therapeutic system containing a first pharmaceutically active substance for the transdermal administration of said first pharmaceutically active substance during a predeterminable period of time, said first pharmaceutically active substance being selected from the group consisting of analgesics, broncholytics, antidiabetics, vasodilators and anti-Parkinson agents; and
b) applying at least one wafer at the beginning of the transdermal administration, wherein said at least one wafer contains said first pharmaceutically active substance or a second or further active substances suitable for the same indication as said first pharmaceutically active substance.

2. The method according to claim 1, further comprising the step of applying at least one further wafer during the period of the transdermal administration, wherein said at least one further wafer contains said first pharmaceutically active substance or a second or further active substances suitable for the same indication as said first pharmaceutically active substance.

3. The method according to claim 1, wherein the patient is a patient where
a rapid or accelerated onset of therapeutic action is required at the beginning or during the period of a long-term therapy; or where
a temporarily increased active substance requirement occurs during the long-term therapy or basic therapy.

4. The method according to claim 1, wherein said first step of the method comprises applying a transdermal therapeutic system which contains said first active substance and applying a wafer which contains said first active substance or a second or further active substances suitable for the same indication.

5. The method according to claim 4, wherein said step of applying a transdermal therapeutic system is repeated at least once after said predeterminable period of time for effecting a long-term therapy or basic medication of the patient by the transdermal administration of said active substance over a prolonged period of time.

6. The method according to claim 5, wherein said further transdermal therapeutic systems containing said active substance are administered at regular intervals selected from the group consisting of 6 hours, 12 hours, 24 hours, 48 hours and 72 hours, for maintaining the long-term therapy or basic therapy for a prolonged period of time.

7. The method according to claim 1 further comprising the step of administrating an additional amount said first pharmaceutically active substance or of a second or further active substance which is suitable for the same indication in the form of a wafer during the period of the transdermal administration or during the long-term therapy, wherein the active substance dose administered by the wafer is suitable for treating an increased active substance requirement of the patient which temporarily occurs during the period of the transdermal administration or during the long-term therapy.

8. The method according to claim 1, wherein said at least one wafer contains an amount of active substance which corresponds to 0.1 to 0.7 times the transdermally administered daily dose.

9. The method according to claim 1, wherein the patient who depends on the administration of said active substance(s) suffers from at least one disease or symptom selected from the group consisting of chronic pain, asthma, diabetes, risk of cardiac infarction, nicotine withdrawal and Parkinson's disease.

10. The method according to claim 3, wherein the temporarily occurring increased active substance requirement is caused by a condition selected from the group consisting of increased pain intensity and breakthrough pain.

11. The method according to claim 1, wherein said first pharmaceutically active substance, or at least one of said second or further active substances, is selected from the group consisting of analgesics, broncholytics, antidiabetics, vasodilators, withdrawal agents and anti-Parkinson agents.

12. The method according to claim 11, wherein said first pharmaceutically active substance, or at least one of said second or further active substances, is selected from the group consisting of the opioids.

13. A combination of medicaments comprising at least one transdermal therapeutic system (TTS) as well as at least one active substance-containing wafer, wherein said at least one transdermal therapeutic system contains a first pharmaceutically active substance, and said at least one active substance-containing wafer contains said first pharmaceutically active substance or a second or further active substances suitable for the same indication as said first pharmaceutically active substance, and wherein said first pharmaceutically active substance is selected from the group consisting of analgesics, broncholytics, antidiabetics, vasodilators and anti-Parkinson agents.

14. The combination according to claim 13, wherein said second or further pharmaceutically active substances are selected from the group consisting of analgesics, broncholytics, antidiabetics, vasodilators and anti-Parkinson agents.

15. The combination according to claim 13, wherein said at least one transdermal therapeutic system enables a systemic, transdermal administration of said active substances contained in said at least one transdermal therapeutic system over a period of at least 24 hours.

16. The combination according to claim 13, wherein said at least one active substance-containing wafer is suitable for oral administration and the therapeutic action starts within 15 minutes following oral administration of said at least one active substance-containing wafer.

17. The combination according to claim 13 wherein said at least one active substance-containing wafer is disintegratable in at least one of an aqueous media and a mucoadhesive.

18. A use of at least one pharmaceutically active substance for manufacturing a combination of medicaments, comprising:
at least one transdermal therapeutic system (TTS) containing a first pharmaceutically active substance selected from the group consisting of analgesics, broncholytics, antidiabetics, vasodilators and anti-Parkinson agents; and
at least one wafer containing said first pharmaceutically active substance or a second or further active substances suitable for the same indication,
for treating a patient who depends on the administration of said first pharmaceutically active substance or said second or further active substances for achieving a long-term therapy or basic therapy.

19. The use according to claim 18, wherein the combination is suitable for treating a patient, where
a rapid or accelerated onset of therapeutic action is required at the beginning or during the period of a long-term therapy; or where
a temporarily increased active substance requirement occurs during the long-term therapy or basic therapy.

20. The use according to claim 18, wherein applying said at least one transdermal therapeutic system effects the long-term therapy or basic medication, and administrating said at least one wafer treats the temporarily increased active substance requirement.

21. The use according to claim 18, wherein the administration of said at least one wafer is carried out when initiating or maintaining a medicinal long-term therapy for bringing about a rapid or accelerated onset of action.

22. The use according to claim 18, wherein the patient who depends on the administration of said active substance(s) suffers from at least one disease or symptom selected from the group consisting of chronic pain, asthma, diabetes, risk of cardiac infarction, nicotine withdrawal and Parkinson's disease.

23. The use according to claim 18, wherein said first pharmaceutically active substance, or said second or further active substances, is selected from the group consisting of analgesics, broncholytics, antidiabetics, vasodilators, withdrawal agents and anti-Parkinson agents.

24. The method according to claim 6, wherein said further transdermal therapeutic systems containing said active substance are administered at regular intervals of 24 hours.

25. The method according to claim 8, wherein said at least one wafer contains an amount of active substance which corresponds to 0.2 to 0.5 times the transdermally administered daily dose.

26. The combination according to claim 15, wherein said at least one transdermal therapeutic system enables a systemic, transdermal administration of said active substances contained in said at least one transdermal therapeutic system over a period of at least 48 hours.

27. The combination according to claim 16, wherein said at least one active substance-containing wafer is suitable for oral administration and the therapeutic action starts within 5 minutes-following oral administration of said at least one active substance-containing wafer.
